(19) United States
(12) Patent Application Publication
(10) Pub. No.: US 2011/0195136 A1
(43) Pub. Date: Aug. 11, 2011

(54) Drop pill for treating coronary heart disease and preparation thereof

(76) Inventors: Jianming Chen, Tianjin (CN); Xijun Yan, Tianjin (CN); Yonghong Zhu, Tianjin (CN); Baoan Gao, Tianjin (CN); Shunnan Zhang, Tianjin (CN); Haiou Dong, Tianjin (CN); Jizhong Zhang, Tianjin (CN); Wei Li, Tianjin (CN); Xinjiang Yan, Tianjin (CN); Ying Zhao, Tianjin (CN)

(21) Appl. No.: 13/122,800

(22) PCT Filed: Sep. 30, 2009
(86) PCT No.: PCT/CN09/74339

§ 371 Date: Apr. 6, 2011

(30) Foreign Application Priority Data
Oct. 6, 2008 (CN) 200810152009.9

(51) Int. Cl. A61K 36/254; A61P 9/00

(52) U.S. Cl. 424/728

(57) ABSTRACT

The present invention relates to a drop pill for treating coronary heart disease, comprising an active pharmaceutical ingredient (API), a matrix adjuvant, a plastifying adjuvant, propylene glycol and water. the API is prepared from Radix salvia miltiorrhira, Panax notoginseng and Borneol; the matrix adjuvant is erythritol; the plastifying adjuvant is one or more selected from the group consisting of polyethylene glycols, xylitol, lactitol, mannitol, glycerine, soluble amylum, gelatin, methyl cellulose, sodium carboxymethycellulose (CMC-Na), hydroxypropyl methylcellulose (HPMC), arabic gum, alginic acid, dextrin, cyclodextrin (CD), citrate, glycerol acetate, dibutyl sebacate, refined coconut oil and castor oil; Wherein, relative to the total weight of the drop pill, the API is 1˜40 wt %, the plastifying adjuvant 0˜10 wt %, the propylene glycol 1˜10 wt %, the water 0˜10 wt % and the balance is the matrix adjuvant. The drop pills of the present invention are safe and non-toxic, low moisture absorption and rapid dissolution.

### FIELD OF THE INVENTION

The present invention relates to the field of pharmaceutical formulation. More specifically, the present invention relates to a drop pill and the preparation thereof.

### BACKGROUND OF THE INVENTION

Cardiovascular and cerebrovascular disease is among the leading causes of death for human being. With the growth in live standard and the aging of the population, the research in pursuit of therapeutic drug for treating cardiovascular and cerebrovascular disease has become a new focus in the field of medicine R&D. CHD is the shortened form of coronary heart disease, a commonly-encountered disease with high incidence of mortality. So, it has been called as the first killer that seriously threatens human's health. Angina pectoris is regarded to be a kind of clinical syndromes caused by coronary artery insufficient blood-supply and acute, transient myocardial ischemia and anoxia. Generally, it is likely to occur at the time of fatigue, over-meal, catching a cold and emotional agitation with the clinical manifestation of sudden onset of retrosternal oppressive pain, compression pain and oppression feeling. In these patients, chief complaints include palpitation, suffocation and sometimes feeling of impending death. For ischemic stroke (known as the “cerebral infarction”), acute attack stage, early stage and recovery stage of the stroke are thought to be good opportunities for preventing and treating it. Hypertension is the most common cardiovascular disease in the world, which has become one of the most widespread epidemics. Usually, it can induce a series of complications by attacking organs of heart, brain and kidney etc, which seriously threatens human's health. According to the statistical data, there have been one hundred million hypertensive patients at present in China, and this number goes up with an annual increase of 300 million or more. Accordingly, how to treat and prevent the aforementioned diseases is of great significance. China has a long history to use Traditional Chinese medicine (TCM) for treating these diseases, especially those single or compound preparations comprising Radix Salvia Miltiorrhiza (RSM) as a main raw material. The single or compound preparations, such as Compound Salvia Tablet, Compound Salvia Injection, Jingzhi Guanxin Capsule and Lemai Capsule etc. have been confirmed to have definite therapeutic effect on these diseases. Among them, the Compound Salvia preparation is focused as a research hotspot in recent years.

Radix Salvia Miltiorrhiza (RSM), a Traditional Chinese medicine (TCM), has a bitter in taste and slightly cold in nature. It demonstrated a series of efficacies, e.g. activating blood circulation by removing blood stasis, relieving uneasiness of mind by nourishing blood, eliminating carbuncle by cooling blood and promoting tissue regeneration by expelling toxin. It is a commonly-used medicine that has been applied in the field of TCM for activating blood circulation by removing blood stasis. Reportedly, in addition to two essential components (diterpenoids-based lipid-soluble components and phenolic acids-based water-soluble components), there are other components comprised in the RSM, e.g. flavonoids, triterpenes and sterols etc. Structurally, the diterpenoids belonging to quinine and ketone include tanshinone I, IIA, II_{R}, V, VI, cryptotanshinone, isotanshinone I, II, II_{B} and dihydrotanshinone I etc, while the water-soluble phenolic acids include Danshensu (Salvianic acid A), protocatechuic aldehyde, protocatechuic acid, caffeic acid, the derivatives of Danshensu and caffeic acid, and depsides by esterification of dimer, e.g. salvianolic acid A, B, C, D, E, G, lithospermic acid B, rosmarinic acid, methyl rosmarinate etc. Among the diterpenoids in the RSM, the tanshinone IIA is one of representative components for activating blood circulation by removing blood stasis. As shown in the modern pharmacological studies, the RSM is confirmed to be effective in dilating coronary artery, antagonizing myocardial ischemia, anti-clotting, resisting thrombosis, relieving by calmness, reducing blood lipid and preventing atherosclerosis.

Panax Notoginseng (PN), a hemostatic herb, is traditionally believed to have the effects of stopping bleed by dissipating blood stasis and reducing bloatedness by relieving pain. Clinically, it is applied for stopping bleed and activating blood circulation. As shown in the modern pharmacological studies, the PN has the effects of both hemostasis and anti-coagulation. The hemostatic effect includes shortening bleeding and coagulation time, increasing platelet count, and making platelets stretch out pseudopodium, aggregate and degranulate, and lowering permeability of capillary etc. Meanwhile, it has been shown that the material basis for anti-coagulation includes a lot of effective fractions extracted from the PN: the total saponins of PN root, the PN panaxadiol type saponins and the PN panaxatriol type saponins. All these components have efficacious actions of inhibiting the platelet aggregation of rabbits and human. Not only can the PN total saponins promote tissue-type plasminogen (t-N) secretion of vascular endothelial cells, but also prevent the formation of thrombosis.

Borneol refers to the crystal processed from Dipterocarpaceae resin (Dipterocarpace). Blumea camphor is the crystal that is processed from leaves of Herba blumeae balsamiferae (Compositae blumea) by steam distillation. Synthesized borneol refers to the product manufactured from camphor or turpentine oil by a chemical synthesis method. Borneol is reported to have the effects of expelling heat by pungency and purging by bitterness, which has been used clinically for relieving stuffiness, dispersing stagnant fire, having a similar efficacy with Musk for waking up a patient from unconsciousness by inducing resuscitation. What is mainly contained in borneol is d-borneol, while in Blumea camphor is I-borneol. As shown in the modern pharmacological studies, borneol has the effects of anti-myocardial ischemia and increasing coronary blood flow. Moreover, borneol can promote blood-brain barrier permeability, thus increasing the capability of drug for crossing the blood-brain barrier.

Compound Salvia Tablet (CST), made of the aforesaid three TCMs, is proven to have the effects of activating blood circulation by removing blood stasis and relieving pain by regulating circulation of Qi. Its indications include Coronary Heart Disease (CHD) and angina pectoris. Compound Salvia Drop pill (CSDP), a drop pill developed from the CST, has better efficacies than those of the CST. Its therapeutic characteristic lies in the rapid onset of action after sublingual administration. Thus, the CSDP is especially suitable for treating acute attack of angina pectoris. In addition, oral administration of the drop pill can also achieve the same effect. The approved actions and indications for the CSDP include activating blood circulation by removing blood stasis and relieving pain by regulating circulation of Qi, used for treating chest distress and angina pectoris. Due to its significant efficacy, the CSDP has been widely applied in clinic.

The reason why both CST and CSDP achieve the same significant effect lies that the above three TCMs of this formulation work compatibly. Although this formulation is composed of the above three TCMs, the effect mechanism of the formulation cannot be briefly explained by the individual function of three constituent herbs, nor can the effect strength be regarded as a simple addition of the three TCMs' efficacies. This formulation is established on the basis of the TCM theory and TCM experiences of activating blood circulation by removing blood stasis, exploiting the advantage of compatibility of the formulation to the full extent. The formulation include strengthening the existing efficacies of the herbs or even making them generate new efficacies, while reducing certain unwanted efficacies, e.g. toxic or side effects. As depicted in the classics of Treatise on Origin and Development of Medicine • Theory of Differences and Similarities between Chinese Medicinal Prescriptions and Herbs by Xu lingyi Lingtai in the Qing Dynasty, “when forming a prescription of TCM, there are two aspects in need of sufficient considerations: making the utmost of positive effects of constituent herbs, while trying best to refraining their side or toxic effects, which has been regarded as where the essence of TCM theory exists, despite the fact that all prescriptions are varied case by case”.

After recent years of clinical application and research, the application scopes of the CSDP have been further expanded. By now, the CSDP has been confirmed to have a lot of treating effects as follows: increasing coronary blood flow, relaxing angio-smooth muscle, promoting collateral circulation establishment, increasing blood oxygen content of the venous sinus, significantly ameliorating acute myocardial ischemia and infarction, increasing anoxic endurance, resisting lipid peroxidation, scavenging harmful free radicals, preventing cells from the injury caused by ischemia and ischemia/re-oxygenation, improving microcirculation, resisting arrhythmia, inhibiting platelet aggregation, promoting the function of fibrinolysis system, preventing thrombosis, reducing blood viscosity, regulating blood lipid, preventing arteriosclerosis, reducing plasma ET content, obviously improving animal's liver, kidney and spleen function, treating chronic hepatic fibrosis, anti-hypertension, anti-oxidation, improving vascular permeability, ameliorating microcirculation disturbance, bettering retinal ischemia and enhancing immune etc, thus the CSDP can be applied to the relevant diseases. As shown in recent years' clinical application, the CSDP has the treating effects not only on cardiovascular and cerebrovascular diseases, but also can achieve the desirable clinical efficacies on microcirculation disturbance-related diseases, e.g. liver dysfunction, kidney dysfunction and diabetic vascular complications etc. Due to the unique features of high efficacy, rapid efficacy, small dose, fixed content of pharmaceutically active component and multi-route of administration, the CSDP is especially suitable for treating CHD and acute attack of angina pectoris, which is deemed to have a better efficacy than that of the CST.

With the worldwide market expansion and the trend of human returning to nature, the reduced side-effects and toxicity, especially the purely natural medicines have increasingly become the first choice. The drop pill belongs to a novel TCM preparation with the characteristics of high efficacy and rapid efficacy, which can effectively overcome the shortcomings and lack of the existing TCM preparations.

However, what universally troubled the drop pill at present is that the adjuvant used in the drop pill has a low degree of naturalness. For a long time, the matrix adjuvant mainly includes polyethylene glycol (PEG), e.g. PEG-4000, PEG-6000 or the mixture thereof, and polyoxyethylene monostearate, poloxamer, polyether and the like are selected occasionally. In terms of quality control (QC), the chemically synthesized materials are the mixtures in a certain range of molecular weight distribution (MWD), which makes it difficult to control the quality of the final drop pill, thus leading to variance in quality from batch to batch. In terms of safety, PEG, as the matrix adjuvant traditionally used for drop pill, can cause gastrointestinal tract stimulation and hemolyzation to some extent, affecting compliance of patients. Additionally, the drop pill prepared by using PEG as the matrix adjuvant is unstable and prone to moisture absorption and conglutination by heating. But an exploration of novel natural materials used as the matrix adjuvant and their preparation methods are difficult because the preparation conditions of drop pill by using up-date common potential natural material substitutes are very strict, and the melting temperature of the matrix adjuvant and dripping conditions of the drop pill are key factors influencing shaping ability of drop pill. Usually, too high melting temperature of the matrix adjuvant will lead to the matrix adjuvant low viscosity and poor plastifying ability; on the contrary, too low temperature makes the drop pill have strong plastifying ability, but the prepared drop pill has shortcomings of being prone to conglutination and deformation etc. Accordingly, it is a very hard work to search a material with a high degree of naturalness as a suitable substitute for the existing matrix adjuvant of drop pills.

Erythritol is a kind of four-carbon polyol with a chemical name of 1, 2, 3, 4-butantetraol and a molecular formula of C₄H₁₀O₄. It is a white and bright powder or crystal, soluble in water, and its aqueous solution is a colorless and non-viscous liquid. As a natural material, erythritol exists widely in fungus (e.g. seaweed and mushroom etc.), fruits (e.g. melon and grape etc.) and various kinds of fermented foods (e.g. grape wine, chewing gum, sake and soy sauce etc). Also, it can be found to exist in human's or animal's tissue or body fluid (e.g. blood, semen and urine). Industrially, erythritol is obtainable by two methods of fermentation or chemical synthesis. Erythritol is a new type of polyol sweetening agent with the advantages as follow: low calorie, good crystallinity, good taste, low moisture adsorption, non-dental caries and causing no gastrointestinal discomfort, and is clinically safe for diabetics when used as the pharmaceutical adjuvant. Meanwhile, it remains extremely stable to acid, heat and fermentation. Under the routine conditions for food industry, browning and decomposition of erythritol has not been observed. Hence, erythritol is called as a “zero-calorie” additive with the familiar taste of sucrose, but contains almost no calorie. After being eaten, it will not lead to the troubles possibly caused by ordinary sugars, e.g. high-calorie (HC), diabetic, and dental caries etc. At present, erythritol has attracted a great attention.

### DETAILED DESCRIPTION OF THE INVENTION

The objective of the present invention is to change the situation that the chemically synthesized materials such as polyethylene glycol (PEG) have been used as the adjuvant for a long time, which leads to the toxic and side effects, poor stability and difficulty in quality control, thus to provide a safe, stable, moisture-proof and good-tasting materials used for developing and producing a drop pill.

Another objective of the present invention is to provide a method for preparing drop pills by using erythritol as the matrix adjuvant.

The objective of the present invention is achieved by the following technical solutions.

A drop pill comprising an active pharmaceutical ingredient (API), a matrix adjuvant, a plastifying adjuvant, propylene glycol and water:

Said API is prepared from Radix salvia miltiorrhira, Panax notoginseng and borneol;
Said matrix adjuvant is erythritol;
Said plastifying adjuvant is one or more selected from the group consisting of polyethylene glycols, xylitol, lactitol, mannitol, glycerine, soluble amylum, gelatin, methyl cellulose, sodium carboxymethycellulose (CMC-Na), hydroxypropyl methylcellulose (HPMC), arabic gum, alginic acid, dextrin, cyclodextrin (CD), citrate, glycerol acetate, dibutyl sebacate, refined coconut oil and castor oil;
Wherein, relative to the total weight of the drop pill, the API is 1˜40 wt %, the plastifying adjuvant 0˜10 wt %, the propylene glycol 1˜10 wt %, the water 0˜10 wt % and the balance is the matrix adjuvant.

According to the present invention, erythritol, used as the matrix adjuvant, can be obtained by a synthesis method, a fermentation method or derived from a natural source. Preferably, erythritol is obtained by a fermentation method or derived from a natural source.

The addition of propylene glycol is for the purpose of increasing solubility of borneol.

According to the present invention, without addition of the plastifying adjuvant, physical properties of drop pills, e.g. spherical degree, shaping ability and rigidity are pharmaceutically acceptable and can meet the clinical requirements. Further, the addition of a proper amount of a plastifying adjuvant can make the final drop pills achieve better effects.

According to the present invention, if the API used is in the form of a liquid extract, no addition of water is needed. On the contrary, if the API used is in the form of a dried extract, the water is required so as to increase the fluidity of the API.

According to the present invention, the API is prepared from a formulation consisting of the crude drugs by weight percentages:

| | |
|---|---|
| Radix salvia miltiorrhira | 63.0%~94.0% |
| Panax notoginseng | 4.0%~35.0% |
| Borneol | 0.5%~2.0%. |

More preferably, the API is prepared from a formulation consisting of the crude drugs by weight percentages:

| | |
|---|---|
| Radix salvia miltiorrhira | 80.0%~90.0% |
| Panax notoginseng | 9.0%~19.0% |
| Borneol | 0.5%~1.7%. |

Most preferably, the API is prepared from a formulation consisting of the crude drugs by weight percentages:

| | |
|---|---|
| Radix salvia miltiorrhira | 82.9% |
| Panax notoginseng | 16.2% |
| Borneol | 0.9%. |

Most preferably, the API is prepared from a formulation consisting of the following crude drugs:

| | |
|---|---|
| Radix salvia miltiorrhira | 31.32 parts by weight |
| Panax notoginseng | 9.21 parts by weight |
| Borneol | 0.50 parts by weight. |

Most preferably, the API is prepared from a formulation consisting of the following crude drugs:

| | |
|---|---|
| Radix salvia miltiorrhira | 59.36 parts by weight |
| Panax notoginseng | 6.38 parts by weight |
| Borneol | 0.34 parts by weight. |

According to the present invention, the API can be prepared by a method comprising steps as follows:
a. the ground crude drugs of Radix salvia miltiorrhira and Panax notoginseng are taken, extracted by heating for 2˜4 times at a temperature of 60˜100° C., an extraction liquid is obtained and filtered; the filtrates are combined and concentrated;
b. the concentrated filtrate is added with ethanol to make a final ethanol content of 50˜85% (volume percentage) and allowed to stand; the obtained supernatant is filtered and concentrated to give a liquid extract with a relative density of 1.15˜1.45 by recovering the ethanol therein.

According to the present invention, the API can be prepared into the form of a liquid extract, or a dried extract which can be prepared from the liquid extract by a conventional method, e.g. spray-drying method. Preferably, the API is prepared into the form of a dried extract.

According to the present invention, the API is preferably 10˜30 wt % of the total weight of the drop pill, more preferably 15˜28 wt % of the total weight of the drop pill.

According to the present invention, the propylene glycol is preferably 2˜6 wt % of the total weight of the drop pill, most preferably 4 wt % of the total weight of the drop pill.

According to the present invention, the plastifying adjuvant is preferably 2˜6 wt % of the total weight of the drop pill.

According to the present invention, a method for preparing the afore-mentioned drop pills is provided, mainly comprising the steps as follows:
(1) an API, a matrix adjuvant, a plastifying adjuvant, propylene glycol and water are blended homogeneously to give a mixture;
(2) the mixture is transferred into a dripping machine and heated to give a melted mixture;
(3) the melted mixture is dripped into a cooling fluid to give drop pills after solidification, and then the drop pills are filtered out;
(4) the cooling fluid on surface of the drop pills is wiped off; and
(5) the wiped drop pills are dried at a low temperature.

Preferably, the API in the afore-mentioned method can be prepared by a method comprising steps as follows:
a. the ground crude drugs of Radix salvia mlltiorrhira and Panax notoginseng are taken, extracted by heating for 2˜4 times at a temperature of 60˜100° C., an extraction liquid is obtained and filtered; the filtrates are combined and concentrated;
b. the concentrated filtrate is added with ethanol to make a final ethanol content of 50˜85% (volume percentage) and allowed to stand; the obtained supernatant is filtered and concentrated to give a liquid extract with a relative density of 1.15˜1.45 by recovering the ethanol therein.

According to the present invention, the API can be prepared into the form of a liquid extract, or a dried extract which can be prepared from the liquid extract by a conventional method, e.g. spray-drying method. Preferably, the API is prepared into the form of a dried extract.

Herein, the melting temperature for melting the mixture in step (2) or the dripping temperature for dripping the melted mixture into the cooling fluid in step (3) is 95˜115° C., preferably 100˜110° C. the cooling fluid is one or more selected from the group consisting of liquid paraffin, methyl silicone oil and vegetable oil, preferably liquid paraffin and/or methyl silicone oil. The temperature of the cooling fluid is −10˜40° C., preferably 2˜10° C.

The drop pill prepared by using erythritol as the matrix adjuvant offers the following advantages:
Excellent safety: no toxic or side effects such as gastrointestinal irritating effect.
Excellent stability: erythritol has low moisture adsorption, good acid-resistance and heat-resistance, helpful for the manufacture and storage of the drop pills.
Enhanced compliance: erythritol, as having a certain sweet, has a good taste, thus enhancing patients' compliance. Also, it is suitable for diabetics, and less prone to dental caries.
Rapid dissolution: the unique features of drop pill with high efficacy and rapid efficacy have been highlighted by rapid dissolution.
Use of erythritol has changed the long-term monotonous situation that only a few kinds of adjuvant have been used in preparing drop pills.

### DESCRIPTION OF DRAWINGS

FIG. 1 is the comparison of moisture adsorption percentages at a relative humidity of 90% between two kinds of the drop pills prepared by using erythritol and PEG as the matrix adjuvant respectively. Herein, ▴ represented the drop pills prepared by using erythritol as the matrix adjuvant; ♦ represented the drop pills prepared by PEG-4000 as the matrix adjuvant.

FIG. 2 is the fingerprint chromatogram of Radix salvia mlltiorrhira comprised in the drop pill prepared by using erythritol as the matrix adjuvant.

FIG. 3 is the fingerprint chromatogram of Radix salvia mlltiorrhira comprised in the extract of Radix salvia mlltiorrhira and Panax notoginseng.

FIG. 4 is the fingerprint chromatogram of Panax notoginseng comprised in the drop pill prepared by using erythritol as the matrix adjuvant.

FIG. 5 is the fingerprint chromatogram of Panax notoginseng comprised in the extract of Radix salvia miltiorrhira and Panax notoginseng.

### EMBODIMENTS

The following examples are given only for the purpose of further illustrating the present invention, but the invention should not be limited by these examples in any way.

### Preparative Example of the Extract

Preparation of a Liquid Extract of Radix Salvia Miltiorrhira and Panax notoginseng in Accordance with the Method Published in Example 1 of Chinese Patent Application No. CN1348815A

Coarsely ground crude drugs of Radix salvia miltiorrhira and Panax notoginseng were placed into an extraction tank, into which 5 times of water as much as the weight of the crude drugs was poured to decoct for 2 hours. After filtration of the decoction, the residue was decocted for 1 hour after adding 4 times of water as much as the weight of the crude drugs for a second extraction. The decoction was filtered and the residue discarded. The filtrates of two times of extraction were combined and concentrated under a reduced pressure to give an extract in a ratio of the extract volume (L) to the weight of the crude drugs (Kg) as 1: (0.9˜1.1). Then, 95% (v/v) ethanol was slowly added into the obtained extract to make a final ethanol content of 69%˜71% (v/v), and allowed to stand for ethanol precipitation for 12 hours, and a supernatant was separated and filtered. The filtrate was concentrated by recovering ethanol to give an extract with a relative density of 1.32˜1.40.

### Example 1

### Formulation

18 g of the liquid extract of Radix salvia miltiorrhira and Panax notoginseng (prepared by the method of the above preparative example of the extract), 1.2 g of borneol, 54 g of erythritol and 3 g of propylene glycol.

### Preparation of the Drop Pills

The erythritol, the liquid extract of Radix salvia miltiorrhira and Panax notoginseng, the borneol and the propylene glycol in the formulation were sufficiently homogenized and poured into a dripping machine. The mixture was heated on a circulating oil-bath to be melted and the temperature of the melted mixture was maintained at 101° C. The melted mixture was dripped into a cooling fluid of methyl silicone oil (5° C.) at a speed of 35 pellets/min. After being well shaped, the drop pills were filtered out and the methyl silicone oil on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 1.96 min without baffle, which complied with the requirements of the Chinese Pharmacopeia.

### Example 2

### Formulation

19.2 g of the dried extract of Radix salvia miltiorrhira and Panax notoginseng (the dried extract was prepared from the liquid extract by conventional spray-drying method, and the liquid extract was prepared by the method of the above preparative example of the extract), 4.8 g of borneol, 32.7 g of erythritol, 2.4 g of propylene glycol and 0.9 g of water.

### Preparation of the Drop Pills

The erythritol, the dried extract of Radix salvia miltiorrhira and Panax notoginseng, the borneol, the propylene glycol and the water in the formulation were sufficiently homogenized and poured into a dripping machine. The mixture was heated on a circulating oil-bath to be melted and the temperature of the melted mixture was maintained at 105° C. The melted mixture was dripped into a cooling fluid of liquid paraffin (7° C.) at a speed of 40 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.1 min without baffle, which complied with the requirements of the Chinese Pharmacopeia.

### Example 3

### Formulation

12 g of the liquid extract of Radix salvia miltiorrhira and Panax notoginseng (prepared by the method of the above preparative example of the extract), 1.2 g of borneol, 36.3 g of erythritol and 0.5 g of propylene glycol.

### Preparation of the Drop Pills

The erythritol, the liquid extract of Radix salvia miltiorrhira and Panax notoginseng, the borneol and the propylene glycol in the formulation were sufficiently homogenized and poured into a dripping machine. The mixture was heated on a circulating oil-bath to be melted and the temperature of the melted mixture was maintained at 103° C. The melted mixture was dripped into a cooling fluid of liquid paraffin (6° C.) at a speed of 45 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.3 min without baffle, which complied with the requirements of the Chinese Pharmacopeia.

### Example 4

### Formulation

12 g of the liquid extract of Radix salvia miltiorrhira and Panax notoginseng (prepared by the method of the above preparative example of the extract), 1.2 g of borneol, 32.8 g of erythritol, 3 g of xylitol and 1 g of propylene glycol.

### Preparation of the Drop Pills

The erythritol, the xylitol, the liquid extract of Radix salvia miltiorrhira and Panax notoginseng, the borneol and the propylene glycol in the formulation were sufficiently homogenized and poured into a dripping machine. The mixture was heated on a circulating oil-bath to be melted and the temperature of the melted mixture was maintained at 108° C. The melted mixture was dripped into a cooling fluid of methyl silicone oil (10° C.) at a speed of 40 pellets/min. After being well shaped, the drop pills were filtered out and the methyl silicone oil on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.2 min without baffle, which complied with the requirements of the Chinese Pharmacopeia.

### Example 5

### Formulation

14.7 g of the dried extract of Radix salvia miltiorrhira and Panax notoginseng (the dried extract was prepared from the liquid extract by conventional spray-drying method, and the liquid extract was prepared by the method of the above preparative example of the extract), 1.2 g of borneol, 52 g of erythritol, 2.5 g of propylene glycol and 1 g of water.

### Preparation of the Drop Pills

The erythritol, the dried extraction of Radix salvia miltiorrhira and Panax notoginseng, the borneol, the propylene glycol and the water in the formulation were sufficiently homogenized and poured into a dripping machine. The mixture was heated on a circulating oil-bath to be melted and the temperature of the melted mixture was maintained at 107° C. The melted mixture was dripped into a cooling fluid of methyl silicone oil (9° C.) at a speed of 38 pellets/min. After being well shaped, the drop pills were filtered out and the methyl silicone oil on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.06 min without baffle, which complied with the requirements of the Chinese Pharmacopeia.

### Example 6

### Formulation

11.7 g of the dried extract of Radix salvia miltiorrhira and Panax notoginseng (the dried extract was prepared from the liquid extract by conventional spray-drying method, and the liquid extract was prepared by the method of the above preparative example of the extract), 1.52 g of borneol, 42.5 g of erythritol, 2.4 g of propylene glycol and 0.9 g of water.

### Preparation of the Drop Pills

The erythritol, the dried extract of Radix salvia miltiorrhira and Panax notoginseng, the borneol, the propylene glycol and the water in the formulation were sufficiently homogenized and poured into a dripping machine. The mixture was heated on a circulating oil-bath to be melted and the temperature of the melted mixture was maintained at 106° C. The melted mixture was dripped into a cooling fluid of liquid paraffin (6° C.) at a speed of 42 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.5 min without baffle, which complied with the requirements of the Chinese Pharmacopeia.

### Example 7

### Formulation

12 g of the liquid extract of Radix salvia miltiorrhira and Panax notoginseng (prepared by the method of the above preparative example of the extract), 1.32 g of borneol, 36 g of erythritol and 2 g of propylene glycol.

### Preparation of the Drop Pills

The erythritol, the liquid extract of Radix salvia miltiorrhira and Panax notoginseng, the borneol and the propylene glycol in the formulation were sufficiently homogenized and poured into a dripping machine. The mixture was heated on a circulating oil-bath to be melted and the temperature of the melted mixture was maintained at 106° C. The melted mixture was dripped into a cooling fluid of liquid paraffin (6° C.) at a speed of 46 pellets/min. After being well shaped, the drop pills were filtered out and the liquid paraffin on the surface of the drop pills was wiped off with absorbent paper and then the drop pills were obtained by drying at a low temperature.

The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.2 min without baffle, which complied with the requirements of the Chinese Pharmacopeia.

### Example 8

### Formulation

0.45 g of the dried extract of Radix salvia miltiorrhira and Panax notoginseng (the dried extract was prepared from the liquid extract by conventional spray-drying method, and the liquid extract was prepared by the method of the above preparative example of the extract), 0.05 g of borneol, 39.5 g of erythritol, 5 g of propylene glycol and 5 g of water.

### Preparation of the Drop Pills

The erythritol, the dried extraction of Radix salvia miltiorrhira and Panax notoginseng, the borneol, the propylene glycol and the water in the formulation were sufficiently homogenized and poured into a dripping machine. The mixture was heated on a circulating oil-bath to be melted and the temperature of the melted mixture was maintained at 115° C. The melted mixture was dripped into a cooling fluid of liquid paraffin (40° C.) at a speed of 43 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.3 min without baffle, which complied with the requirements of the Chinese Pharmacopeia.

### Example 9

### Formulation

16.2 g of the dried extract of Radix salvia miltiorrhira and Panax notoginseng (the dried extract was prepared from the liquid extract by conventional spray-drying method, and the liquid extract was prepared by the method of the above preparative example of the extract), 1.8 g of borneol, 36 g of erythritol, 2.5 g of propylene glycol and 1.5 g of water.

### Preparation of the Drop Pills

The erythritol, the dried extract of Radix salvia miltiorrhira and Panax notoginseng, the borneol, the propylene glycol and the water in the formulation were sufficiently homogenized and poured into a dripping machine. The mixture was heated on a circulating oil-bath to be melted and the temperature of the melted mixture was maintained at 105° C. The melted mixture was dripped into a cooling fluid of liquid paraffin (5° C.) at a speed of 38 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.1 min without baffle, which complied with the requirements of the Chinese Pharmacopeia.

### Example 10

### Formulation

21.42 g of the dried extract of Radix salvia miltiorrhira and Panax notoginseng (the dried extract was prepared from the liquid extract by conventional spray-drying method, and the liquid extract was prepared by the method of the above preparative example of the extract), 2.38 g of borneol, 39.5 g of erythritol, 2.7 g of propylene glycol and 2 g of water

### Preparation of the Drop Pills

The erythritol, the dried extract of Radix salvia miltiorrhira and Panax notoginseng, the borneol, the propylene glycol and the water in the formulation were sufficiently homogenized and poured into a dripping machine. The mixture was heated on a circulating oil-bath to be melted and the temperature of the melted mixture was maintained at 103° C. The melted mixture was dripped into a cooling fluid of liquid paraffin (4° C.) at a speed of 40 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.6 min without baffle, which complied with the requirements of the Chinese Pharmacopeia.

### Example 11

### Formulation

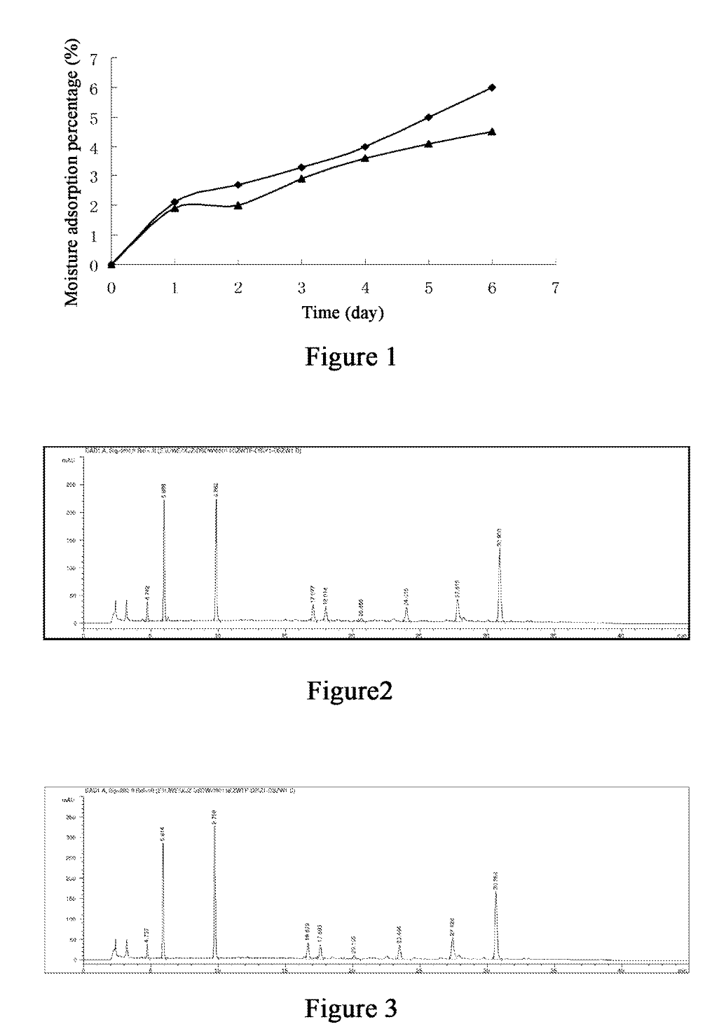

Comparison test had been made between two kinds of drop pills prepared by the method described in Example 2 by using erythritol and PEG-4000 as the matrix adjuvant respectively. The moisture adsorption percentages between the two kinds of drop pills at a relative humidity of 90% are compared (Guideline of test for hygroscopicity according to the appendix XI X Jin Chinese Pharmacopeia (2005)), the results are shown in FIG. 1. It can be seen that, compared with the hygroscopicity of the drop pills prepared by using PEG-4000, the drop pills prepared by using erythritol have significantly low hygroscopicity, which is helpful for the manufacture and storage of the drop pills.

### Example 12

### Formulation

12 g of the liquid extract of Radix salvia miltiorrhira and Panax notoginseng (prepared by the method of the above preparative example of the extract), 1.2 g of borneol, 42 g of erythritol and 2 g of propylene glycol.

### Preparation of the Drop Pills

The erythritol, the liquid extract of Radix salvia miltiorrhira and Panax notoginseng, the borneol and the propylene glycol in the formulation were sufficiently homogenized and poured into a dripping machine. The mixture was heated on a circulating oil-bath to be melted and the temperature of the melted mixture was maintained at 95° C. The melted mixture was dripped into a cooling fluid of liquid paraffin (2° C.) at a speed of 46 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.2 min without baffle, which complied with the requirements of the Chinese Pharmacopeia.

### Example 13

### Formulation

21 g of the dried extract of Radix salvia miltiorrhira and Panax notoginseng (the dried extract was prepared from the liquid extract by conventional spray-drying method, and the liquid extract was prepared by the method of the above preparative example of the extract), 2 g of borneol, 63 g of erythritol, 2.6 g of propylene glycol and 2.6 g of water.

### Preparation of the Drop Pills

The erythritol, the dried extract of Radix salvia miltiorrhira and Panax notoginseng, the borneol, the propylene glycol and the water in the formulation were sufficiently homogenized and poured into a dripping machine. The mixture was heated on a circulating oil-bath to be melted and the temperature of the melted mixture was maintained at 108° C. The melted mixture was dripped into a cooling fluid of liquid paraffin (−10° C.) at a speed of 38 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.8 min without baffle, which complied with the requirements of the Chinese Pharmacopeia.

### Example 14

### Formulation

20 g of the liquid extract of Radix salvia miltiorrhira and Panax notoginseng (prepared by the method of the above preparative example of the extract), 1.76 g of borneol, 48 g of erythritol, 8 g of soluble amylum and 3 g of propylene glycol.

### Preparation of the Drop Pills

The erythritol, the liquid extract of Radix salvia miltiorrhira and Panax notoginseng, the borneol, the soluble amylum and the propylene glycol in the formulation were sufficiently homogenized and poured into a dripping machine. The mixture was heated on a circulating oil-bath to be melted and the temperature of the melted mixture was maintained at 100° C. The melted mixture was dripped into a cooling fluid of soybean oil (6° C.) at a speed of 42 pellets/min. After being well shaped, the drop pills were filtered out and the soybean oil on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.4 min without baffle, which complied with the requirements of the Chinese Pharmacopeia.

### Example 15

### Formulation

19.5 g of the dried extract of Radix salvia miltiorrhira and Panax notoginseng (the dried extract was prepared from the liquid extract by conventional spray-drying method, and the liquid extract was prepared by the method of the above preparative example of the extract), 2.3 g of borneol, 66 g of erythritol, 7.3 g of mannitol, 4 g of propylene glycol and 1.5 g of water.

### Preparation of the Drop Pills

The erythritol, the dried extract of Radix salvia miltiorrhira and Panax notoginseng, the borneol, the mannitol, the propylene glycol and the water in the formulation were sufficiently homogenized and poured into a dripping machine. The mixture was heated on a circulating oil-bath to be melted and the temperature of the melted mixture was maintained at 105° C. The melted mixture was dripped into a cooling fluid of liquid paraffin (5° C.) at a speed of 44 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.6 min without baffle, which complied with the requirements of the Chinese Pharmacopeia.

### Example 16

### Formulation

20 g of the liquid extract of Radix salvia miltiorrhira and Panax notoginseng (prepared by the method of the above preparative example of the extract), 1.76 g of borneol, 163.02 g of erythritol, 21.76 g of arabic gum and 4.22 g of propylene glycol.

### Preparation of the Drop Pills

The erythritol, the liquid extract of Radix salvia miltiorrhira and Panax notoginseng, the borneol, the arabic gum and the propylene glycol in the formulation were sufficiently homogenized and poured into a dripping machine. The mixture was heated on a circulating oil-bath to be melted and the temperature of the melted mixture was maintained at 102° C. The melted mixture was dripped into a cooling fluid of liquid paraffin (6° C.) at a speed of 46 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.8 min without baffle, which complied with the requirements of the Chinese Pharmacopeia.

### Example 17

### Formulation

20 g of the liquid extract of Radix salvia miltiorrhira and Panax notoginseng (prepared by the method of the above preparative example of the extract), 1.76 g of borneol, 111.64 g of erythritol, 2.9 g of sodium carboxymethycellulose and 8.7 g of propylene glycol.

### Preparation of the Drop Pills

The erythritol, the liquid extract of Radix salvia miltiorrhira and Panax notoginseng, the borneol, the sodium carboxymethycellulose and the propylene glycol in the formulation were sufficiently homogenized and poured into a dripping machine. The mixture was heated on a circulating oil-bath to be melted and the temperature of the melted mixture was maintained at 106° C. The melted mixture was dripped into a cooling fluid of liquid paraffin (8° C.) at a speed of 39 pellets/min. After being well shaped, the drop pills were filtered out and the liquid paraffin on the surface of the drop pills was wiped off with absorbent paper and then the drop pills were obtained by drying at a low temperature. The results indicated that the obtained drop pills were sphere in even size, uniform color and without conglutination. Determination result of disintegration time according to the method described in Chinese Pharmacopeia (2005) revealed that the drop pills passed the wire mesh completely within a mean time of 2.3 min without baffle, which complied with the requirements of the Chinese Pharmacopeia.

### Example 18

The fingerprint chromatogram of Radix salvia miltiorrhira was assayed in the drop pills prepared in Example 2 by using erythritol as the matrix adjuvant (see FIG. 2), and compared with the fingerprint chromatogram of Radix salvia miltiorrhira in the extract of Radix salvia miltiorrhira and Panax notoginseng (see FIG. 3). As shown in the results, the similarity degree of both fingerprint chromatograms of Radix salvia miltiorrhira was more than 90%.

### Determination of the Fingerprint Chromatogram of Radix Salvia Miltiorrhira

HPLC conditions:

### Chromatographic Column: Agilent SB-C₁₈ (4.6×250 mm, 5 μm);

Flow rate: 1.0 ml/min;
Detecting wavelength: 280 nm;
Column temperature: 30° C.;
Injection volume: 10 μl;
Mobile phase: phase A: 0.02% phosphoric acid aqueous solution; phase B: 80% (v/v) acetonitrile/water containing 0.02% phosphate acid

| | phase A | phase B |
|---|---|---|
| 0 min | 90% | 10% |
| 8 min | 78% | 22% |
| 15 min | 74% | 26% |
| 35 min | 61% | 39% |
| 40 min | 90% | 10% |
| 50 min | 90% | 10% |

### Preparation of Sample:

250 mg of the drop pills prepared in Example 2 by using erythritol as the matrix adjuvant was placed into a 10 ml volumetric flask, into which a proper amount of distilled water was added, the drop pills were dissolved by ultrasonication for 15 min. Then, water was added to the calibration line. The obtained solution was filtered to give the sample.

### Example 19

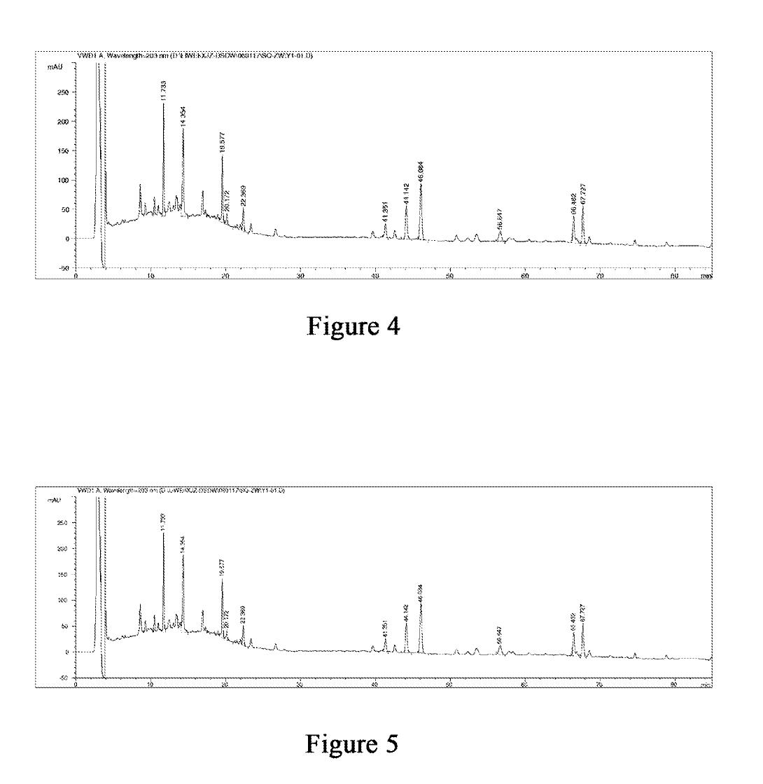

The fingerprint chromatogram of Panax notoginseng was assayed in the drop pills prepared in Example 2 by using erythritol as the matrix adjuvant (see FIG. 4), and compared with the fingerprint chromatogram of Panax notoginseng in the extract of Radix salvia mlltiorrhira and Panax notoginseng (see FIG. 5). As shown in the results, the similarity degree of both fingerprint chromatograms of Panax notoginseng was more than 90%.

Determination of the Fingerprint Chromatogram of Panax notoginseng
HPLC conditions:

### Chromatographic Column: Agilent SB-C₁₈ (4.6×250 mm, 5 μm);

Flow rate: 0.8 ml/min;
Detecting wavelength: 203 nm;
Column temperature: 30° C.;
Injection volume: 20 μl;
Mobile phase: phase A: 0.01% acetic acid aqueous solution; phase B: acetonitrile containing 0.01% acetic acid

| | phase A | phase B |
|---|---|---|
| 0 min | 80% | 20% |
| 15 min | 65% | 35% |
| 25 min | 65% | 35% |
| 40 min | 57% | 43% |
| 50 min | 57% | 43% |
| 65 min | 42% | 58% |
| 75 min | 25% | 75% |
| 80 min | 80% | 20% |
| 85 min | 80% | 20% |

### Preparation of Samples:

10 ml of aqueous ammonia (4%) was added into 1.0 g of the drop pills prepared in Example 2 by using erythritol as the matrix adjuvant to dissolve by ultrasonication. The solution was filtered through 0.45 μm filter membrane, and 5 ml of the filtrate was passed through C₁₈ small column. The column was washed with 10 ml water, and the eluent of water was discarded. The column was then washed with methanol and an eluent with a volume of 5 ml was obtained. The eluent of methanol was filtered to give the sample.

### Example 20

The drop pills by using erythritol as the matrix adjuvant and the drop pills by using PEG-4000 as the matrix adjuvant were prepared according to the method of Example 2. The disintegration times of the above two kinds of the drop pills were measured according to the method described in Chinese Pharmacopeia (2005) and compared. The results indicated that the disintegration time of the drop pills using erythritol as the matrix adjuvant was 2.1 min, while the drop pills by using PEG-4000 as the matrix adjuvant was 6.2 min. It was revealed that the disintegration of the drop pills prepared by using erythritol was remarkably rapid than that of the drop pills prepared by using PEG-4000. This well reflects the advantages and features of the drop pills, i.e. high efficacy and rapid efficacy.

In order to better understand the present invention, hereafter the advantages of the present invention were further explained by a way of some experiments, such as disintegration time, weight variation, rigidity and viscidity of the drop pills prepared by matrix adjuvant used in Example 2 of the present invention.

### Experimental Example 1

### Comparative Experimental Example of Disintegration Time and Weight Variation

In comparing the drop pills prepared by using the matrix adjuvant of this invention with those prepared by using polyethylene glycol respectively, the disintegration time was measured to determine whether the drop pills of the present invention have satisfactory release effect, and the indexes such as the weight variation were measured to determine whether the preparation process is mature and adaptable to the industrial applicability.

### 1. Samples:

The drop pills prepared in Example 2 of the present invention (hereinafter labeled as “new”) and the drop pills prepared by the method described in Example 1 of Chinese Patent No. CN1348815A (hereinafter labeled as “known”). The method of Example 1 in Chinese Patent No. CN1348815A was as follows: 11.7 g of the extract of Radix salvia miltiorrhira and Panax notoginseng of Example 1, 1.38 g of borneol and 18 g of PEG-6000 were mixed well and heated to 85˜90° C.; after the mixture was melted for 20˜120 min, the melted mixture was transferred into a dripping tank at a temperature of 85˜90° C., and the melted mixture was dripped into the liquid paraffin with a temperature of 7˜8° C.; the drop pills were taken out, and the liquid paraffin on the surface of the drop pills was removed. The drop pills were obtained by selecting drop pills with a wire mesh.

### 2. Method and Results:

Disintegration time: measured according to the method of the corresponding item in Chinese Pharmacopoeia; weight variation: measured according to the method of the corresponding item in Chinese Pharmacopoeia. Results are shown in Table 1.

TABLE 1
Comparison of disintegration time and weight variation of three batches of the drop pills
between the drop pills prepared by using the novel matrix adjuvant (labeled as “new”)
with those prepared by using PEG as the main adjuvant (labeled as “known”)

| | | 0 | 1^{st} | 2^{nd} | 3^{rd} | | 12^{th} | 18^{th} |
|---|---|---|---|---|---|---|---|---|
| | | month | month | month | month | 6^{th} month | month | month |
| Criterion | | Results | | | | | | |
| 1^{st} | Weight variation | within | within | within | within | within | within | within |
| batch | (±15%) | ±10% | ±10% | ±10% | ±10% | ±10% | ±10% | ±10% |
| | Disintegration time | | | | | | | |
| | (new) | 2′05″ | 2′09″ | 2′16″ | 2′15″ | 2′16″ | 2′20″ | 2′23″ |
| | (known) | 5′11″ | 5′06″ | 5′15″ | 5′19″ | 5′26″ | 5′16″ | 5′35″ |
| 2^{nd} | Weight variation | within | within | within | within | within | within | within |
| batch | (±15%) | ±10% | ±10% | ±10% | ±10% | ±10% | ±10% | ±10% |
| | Disintegration time | | | | | | | |
| | (new) | 1′57″ | 1′59″ | 1′56″ | 2′04″ | 2′09″ | 2′10″ | 2′08″ |
| | (known) | 5′14″ | 5′18″ | 5′21″ | 5′19″ | 5′26″ | 5′34″ | 5′32″ |
| 3^{rd} | Weight variation | within | within | within | within | within | within | within |
| batch | (±15%) | ±10% | ±10% | ±10% | ±10% | ±10% | ±10% | ±10% |
| | Disintegration time | | | | | | | |
| | (new) | 2′12″ | 2′09″ | 2′15″ | 2′13″ | 2′17″ | 2′20″ | 2′25″ |
| | (known) | 5′10″ | 5′17″ | 5′21″ | 5′23″ | 5′26″ | 5′30″ | 5′37″ |

The experiment data indicated that the disintegration times of the drop pills prepared by the novel matrix adjuvant is shorter than those prepared by PEG as the main adjuvant, and that the weight variation of both of the new or known drop pills are controlled within the range prescribed in Chinese Pharmacopoeia. The experiment data also indicated the disintegration speed of the drop pills prepared by the novel matrix adjuvant is more rapid and more favorable for making API take effect in a time as short as possible. The weight variation of both of the new or known drop pills are controlled within the range prescribed in Chinese Pharmacopoeia, the variation between them is not notable in statistics. Therefore, the natural matrix adjuvant can replace the current chemically synthesized adjuvants for industrial production.

### Experimental Example 2

### Comparative Experimental Example of Rigidity and Viscidity of the Drop Pills Between Those Prepared by Using the Matrix Adjuvants of the Present Invention and Those Prepared by Using PEG as the Main Matrix Adjuvant

### 1. Samples:

The drop pills prepared in Example 2 of the present invention (hereinafter labeled as “new”) and the drop pills prepared by the method described in Example 1 of Chinese Patent No. CN1348815A (hereinafter labeled as “known”). The method of Example 1 in Chinese Patent No. CN1348815A was as follows: 11.7 g of the extract of Radix salvia miltiorrhira and Panax notoginseng of Example 1, 1.38 g of borneol and 18 g of PEG-6000 were mixed well and heated to 85˜90° C.; after the mixture was melted for 20˜120 min, the melted mixture was transferred into a dripping tank at a temperature of 85˜90° C., and the melted mixture was dripped into the liquid paraffin with a temperature of 7˜8° C.; the drop pills were taken out, and the liquid paraffin on the surface of the drop pills was removed. The drop pills were obtained by selecting drop pills with a wire mesh.

### 2. Method and Results:

Three batches of the drop pills were taken, placed into porcelain bottles respectively and sealed tightly with bottle stoppers; the sealed bottles were placed into a desiccator with a saturated NaCl solution (humidity 75%) in its bottom, and then the desiccator was put into a drying cabinet at a constant temperature of 40° C. Samples were collected at regular intervals to examine the rigidity and viscidity of the drop pills. Results are shown in Table 2.

TABLE 2
Comparison of characters of the three batches of the drop pills between the drop pills
prepared by using the novel matrix adjuvant (labeled as “new”) with those prepared by using
PEG as the main adjuvant (labeled as “known”)

| | | 0 month | 1^{st} month | 2^{nd} month | 3^{rd} month | 6^{th} month | 12^{th} month | 18^{th} month |
|---|---|---|---|---|---|---|---|---|
| Criterion | | Results | | | | | | |
| 1^{st} | viscidity | * (known) | * (known) | * (known) | * (known) | ** (known) | ** (known) | *** (known) |
| batch | | * (new) | * (new) | * (new) | * (new) | * (new) | ** (new) | ** (new) |
| | rigidity | A (known) | A (known) | A (known) | A (known) | B (known) | C (known) | C (known) |
| | | A (new) | A (new) | A (new) | A (new) | A (new) | A (new) | C (new) |
| 2^{nd} | viscidity | * (known) | * (known) | * (known) | * (known) | * (known) | ** (known) | ** (known) |
| batch | | * (new) | * (new) | * (new) | * (new) | * (new) | * (new) | ** (new) |
| | rigidity | A (known) | A (known) | A (known) | A (known) | B (known) | C (known) | C (known) |
| | | A (new) | A (new) | A (new) | A (new) | A (new) | A (new) | C (new) |
| 3^{rd} | viscidity | * (known) | * (known) | * (known) | * (known) | ** (known) | ** (known) | *** (known) |
| batch | | * (new) | * (new) | * (new) | * (new) | * (new) | * (new) | ** (new) |
| | rigidity | A (known) | A (known) | A (known) | A (known) | A (known) | C (known) | C (known) |
| | | A (new) | A (new) | A (new) | A (new) | A (new) | A (new) | C (new) |

Note:
* = not sticky;
** = slightly sticky;
*** = sticky;
A = Hard;
B = less hard than normal;
C = much less hard than normal.

The experiment data indicated that, compared with the drop pills prepared by using PEG, the rigidity variation of the drop pills prepared by using the novel matrix adjuvant is similar or slightly more rigid. The viscidity variation between them is similar. That is, the rigidity variation and viscidity variation between the new and known drop pills are similar. Therefore, the natural matrix adjuvant can replace the current chemically synthesized adjuvants for industrial production.

### Experimental Example 3

### Comparative Experimental Example of Heat Resistance

Equal amount of the drop pills prepared by two different kinds of matrix adjuvants were weighed respectively and placed into stopped glass bottles to examine their dumpling fluidity at different temperatures. The heat resistance of the drop pill was reflected by its fluidity. The heat resistance was poor if the drop pills had a poor fluidity, conglutinated into block or melted. On the contrary, the heat resistance of the drop pills was good if the drop pill had a good fluidity and can flow freely. Hence, the dumpling fluidity is always used to evaluate the heat resistance of the drop pills. The results are shown in Table 3.

TABLE 3
Comparison of heat resistance

| Sample | T (° C.) | dumpling fluidity |
|---|---|---|
| new | 50 | good dumpling fluidity, freely flowing |
| (erythritol) | 55 | good dumpling fluidity, freely flowing |
| | 60 | good dumpling fluidity, freely flowing |
| known | 50 | poor dumpling fluidity, some conglutinating |
| (PEG-6000) | 55 | poor dumpling fluidity, conglutinating to block |
| | 60 | totally melted into a cake |

The results indicated, compared with the drop pills prepared by using PEG, the heat resistance of the drop pill prepared by using erythritol as the matrix adjuvant is better. Therefore, the natural matrix adjuvant can not only facilitate storage and production of the drop pills at a high temperature, but can prevent the drop pills from conglutination caused by the high temperature, helping patients' administration of the drop pills.

1-19. (canceled)

20. A drop pill for treating coronary heart disease, comprising an active pharmaceutical ingredient (API), a matrix adjuvant, a plastifying adjuvant, propylene glycol and water:
said API is prepared from Radix salvia miltiorrhira, Panax notoginseng and borneol;
said matrix adjuvant is erythritol;
said plastifying adjuvant is one or more selected from the group consisting of polyethylene glycols, xylitol, lactitol, mannitol, glycerine, soluble amylum, gelatin, methyl cellulose, sodium carboxymethycellulose, hydroxypropyl methylcellulose, arabic gum, alginic acid, dextrin, cyclodextrin, citrate, glycerol acetate, dibutyl sebacate, refined coconut oil and castor oil;
wherein, relative to the total weight of the drop pill, the active pharmaceutical ingredient is 1-40 wt %, the plastifying adjuvant 0-10 wt %, the propylene glycol 1-10 wt %, the water 0-10 wt % and the balance is the matrix adjuvant.

21. The drop pill according to claim 20, wherein relative to the total weight of the drop pill, the active pharmaceutical ingredient is 10-30 wt %.

22. The drop pill according to claim 21, wherein relative to the total weight of the drop pill, the active pharmaceutical ingredient is 15-28 wt %.

23. The drop pill according to claim 20, wherein relative to the total weight of the drop pill, the propylene glycol is 2-6 wt %.

24. The drop pill according to claim 23, wherein relative to the total weight of the drop pill, the propylene glycol is 4 wt %.

25. The drop pill according to claim 20, wherein relative to the total weight of the drop pill, the plastifying adjuvant is 2-6 wt %.

26. The drop pill according to claim 20, wherein relative to the total weight of the drop pill, the water is 0-3 wt %.

27. The drop pill according claim 20, wherein the active pharmaceutical ingredient is prepared from a formulation consisting of the crude drugs by weight percentages:
Radix salvia miltiorrhira: 63.0-94.0%
Panax notoginseng: 4.0-35.0%
Borneol: 0.5-2.0%.

28. The drop pill according to claim 27, wherein the active pharmaceutical ingredient is prepared from a formulation consisting of the crude drugs by weight percentages:
Radix salvia miltiorrhira: 80.0-90.0%
Panax notoginseng: 9.0-19.0%
Borneol: 0.5-1.7%.

29. The drop pill according to claim 27, wherein the active pharmaceutical ingredient is prepared from a formulation consisting of the crude drugs by weight percentages:
Radix salvia miltiorrhira: 82.9%
Panax notoginseng: 16.2%
Borneol: 0.9%.

30. The drop pill according to claim 27, wherein the active pharmaceutical ingredient is prepared from a formulation consisting of the following crude drugs:
Radix salvia miltiorrhira: 31.32 parts by weight
Panax notoginseng: 9.21 parts by weight
Borneol: 0.50 parts by weight.

31. The drop pill according to claim 27, wherein the active pharmaceutical ingredient is prepared from a formulation consisting of the following crude drugs:
Radix salvia miltiorrhira: 59.36 parts by weight
Panax notoginseng: 6.38 parts by weight
Borneol: 0.34 parts by weight.

32. The drop pill according to claim 20, wherein the active pharmaceutical ingredient is prepared by a method comprising the steps as follows:
a. the ground crude drugs of Radix salvia miltiorrhira and Panax notoginseng are taken, extracted by heating for 2-4 times at a temperature of 60-100° C., an extraction liquid is obtained and filtered; the filtrates are combined and concentrated; and
b. the concentrated filtrate is added with ethanol to make a final ethanol content of 50-85% (volume percentage) and allowed to stand; the obtained supernatant is filtered and concentrated to give a liquid extract with a relative density of 1.15-1.45 by recovering the ethanol therein.

33. The drop pill according to claim 27, wherein the active pharmaceutical ingredient is prepared by a method comprising the steps as follows:
a. the ground crude drugs of Radix salvia miltiorrhira and Panax notoginseng are taken, extracted by heating for 2-4 times at a temperature of 60-100° C., an extraction liquid is obtained and filtered; the filtrates are combined and concentrated; and
b. the concentrated filtrate is added with ethanol to make a final ethanol content of 50-85% (volume percentage) and allowed to stand; the obtained supernatant is filtered and concentrated to give a liquid extract with a relative density of 1.15-1.45 by recovering the ethanol therein.

34. The drop pill according to claim 32, wherein the method further comprises a step of preparing a dried extract by spray-drying of the liquid extract.

35. The drop pill according to claim 33, wherein the method further comprises a step of preparing a dried extract by spray-drying of the liquid extract.

36. A method for preparing any drop pills of claim 20, comprising the steps as follows:
(1) an active pharmaceutical ingredient, a matrix adjuvant, a plastifying adjuvant, propylene glycol and water are blended homogeneously to give a mixture;
(2) the mixture is transferred into a dripping machine and heated to give a melted mixture;
(3) the melted mixture is dripped into a cooling fluid to give drop pills after solidification, and then the drop pills are filtered out;
(4) the cooling fluid on the surface of the drop pills is wiped off; and
(5) the wiped drop pills are dried at a low temperature.

37. The method according to claim 36, wherein the active pharmaceutical ingredient is prepared by a method comprising the steps as follows:
a. the ground crude drugs of Radix salvia miltiorrhira and Panax notoginseng are taken, extracted by heating for 2-4 times at a temperature of 60-100° C., an extraction liquid is obtained, and the extraction liquid is filtered; the filtrates are combined and concentrated; and
b. the concentrated filtrate is added with ethanol to make a final ethanol content 5085% (volume percentage) and allowed to stand; the obtained supernatant is filtered and concentrated to give a liquid extract with a relative density of 1.15-1.45 by recovering the ethanol therein.

38. The method according to claim 37, wherein the method for preparing the active pharmaceutical ingredient further comprises a step of preparing a dried extract by spray-drying the liquid extract.

39. The method according to claim 38, wherein a melting temperature for melting the mixture in step (2) or a dripping temperature for dripping the melted mixture into the cooling fluid in step (3) is 95-115° C.; the cooling fluid is one or more selected from liquid paraffin, methyl silicone oil and vegetable oil, and the temperature of the cooling fluid is −10-40° C.
